(19) United States
(12) Patent Application Publication
(10) Pub. No.: US 2026/0036599 A1
(43) Pub. Date: Feb. 5, 2026

(54) RATIO BETWEEN LTA4H AND METRNL IN THE ASSESSMENT OF POLYCYSTIC OVARIAN SYNDROME

(71) Applicant: Roche Diagnostics Operations, Inc., Indianapolis, IN (US)

(72) Inventors: Deirdre Mary Allegranza, Küsnacht (CH); Annunziata Di Domenico, Kilchberg (CH); Martin Hund, Horw (CH); Martin Klammer, Muenchen (DE); Anika Mang, Holzkirchen (DE); Johanna Carolina Sillman, Zug (CH)

(21) Appl. No.: 18/997,730

(22) PCT Filed: Jul. 20, 2023
(86) PCT No.: PCT/EP2023/070093

§ 371 Date: Jan. 22, 2025

(30) Foreign Application Priority Data
Jul. 22, 2022 (EP) 22186518.1

(51) Int. Cl. G01N 33/88

(52) U.S. Cl. CPC G01N 33/88; C12Y 303/02006; G01N 2333/914; G01N 2470/06; G01N 2800/042; G01N 2800/048; G01N 2800/361; G01N 2800/367; G01N 2800/52

(57) ABSTRACT

The present invention relates to a method for diagnosing Polycystic Ovarian Syndrome (PCOS) in a subject, said method comprising the steps of a) determining the amount or concentration of total LTA4H in sample from the subject, b) determining the amount or concentration of METRNL in a sample from the subject, c) calculating a score of the amounts or concentration determined in steps a) and b), d) comparing the calculated score with a reference score, and e) diagnosing PCOS in a subject.

The present invention relates to a method for diagnosing PCOS in a subject, said method comprising the steps of a) determining the amount or concentration of LTA4H in sample from the subject, b) determining the amount or concentration of METRNL in a sample from the subject, c) calculating a score of the amounts or concentrations determined in steps a) and b), d) comparing the calculated score with a reference score, and e) diagnosing PCOS in a subject. Further it relates to a method of selecting a patient for therapy of PCOS, said method comprising a) determining the amount or concentration of LTA4H in sample from the subject, b) determining the amount or concentration of METRNL in a sample from the subject, c) calculating a score of the amounts or concentration determined in steps a) and b), d) comparing the calculated score with a reference score, and e) selecting the patient for PCOS therapy. Further it relates to a method for monitoring PCOS progression in a subject having PCOS or for monitoring response to treatment in a subject having PCOS, said method comprising a) determining the amount or concentration of LTA4H in sample from the subject, b) determining the amount or concentration of METRNL in a sample from the subject, c) calculating a score of the amounts or concentrations determined in steps a) and b), d) comparing the calculated score with a reference score, and e) monitoring progression in the subject suffering from PCOS or being treated for PCOS. Finally the present invention relates to a computer-implemented method for the diagnosis of PCOS in a subject.

### BACKGROUND OF THE INVENTION

Polycystic ovarian syndrome (PCOS) is a heterogeneous gynecological condition that is defined by a combination of signs of androgen excess and ovarian dysfunction. Patients with PCOS can have a range of clinical presentations, which can be of reproductive and/or of metabolic type. Reproductive presentations include irregular menstrual cycle, infertility, pregnancy complications and hirsutism, whereas metabolic presentations include obesity, insulin resistance, metabolic syndrome, pre-diabetes, type-2 diabetes and cardiovascular factors. These clinical presentations are also associated with psychological disorders, such as anxiety and depression (Escobar-Morreale, H. F. 2018; International evidence-based guideline for the assessment and management of polycystic ovary syndrome 2018).

The symptoms are not specific for PCOS and often patients are diagnosed only after longer evaluations for infertility. For a final diagnosis of PCOS, other conditions or diseases should be excluded such as pregnancy, non-classical adrenal hyperplasia (NCAH), Congenital Adrenal Hyperplasia, androgen secreting tumors, Cushing syndrome, thyroid disorders, or hyperprolactinemia (Escobar-Morreale H F. Polycystic ovary syndrome: definition, aetiology, diagnosis and treatment. Nat Rev Endocrinol. 2018; 14(5):270-284; Teede H J, Misso M L, Costello M F, et al. International PCOS Network. Recommendations from the international evidence-based guideline for the assessment and management of polycystic ovary syndrome. Hum Reprod. 2018; 33(9):1602-1618). Diagnostic tests which may be used to exclude other diseases are e.g.
17alpha-Hydroxyprogesterone (17-OHP) to exclude NCAH (Nordenstrom and Falhammar 2018)
Prolactin to exclude hyperprolactinemia
Cortisol to exclude patients suffering from Cushing's syndrome
Thyroid Stimulating Hormone (TSH) to exclude thyroid disorders

PCOS may be caused by a combination of genetic, epigenetic and environmental factors, such as inheritance.

Despite the fact that PCOS is one of the most common endocrine disorders in women, affecting 10% of the women during their reproductive years, up to 70% of the affected women remain undiagnosed (March W A, Moore V M, Willson K J, et al. The prevalence of polycystic ovary syndrome in a community sample assessed under contrasting diagnostic criteria. Hum Reprod. 2010; 25(2):544-51).

The mostly widely used criteria for PCOS diagnosis are the so-called Rotterdam Criteria. PCOS is indicated, if at least 2 of the following criteria apply: (i) irregular cycles (oligomenorrhea) and/or ovulatory dysfunction (oligo-anovulation, OA), (ii) clinical and/or biochemical hyperandrogenism (HA) and (iii) polycystic ovarian morphology (PCOM) (PCOS Consensus Workshop Group, Fertil Steril 2004; 81:19-25). The first criterion is defined as menstrual cycles with a cycle length of less than 21 days or more than 35 days or less than 8 cycles per year. Clinical and/or biochemical signs of hyperandrogenism, where clinical hyperandrogenism is defined as hirsutism (excess and male-pattern hair growth) and/or acne, and biochemical hyperandrogenism is defined as higher levels of free androgens compared to healthy controls. Clinical hyperandrogenism is also defined as a modified Ferriman-Gallwey score of >8. Biochemical hyperandrogenism may be assessed using free testosterone or the free androgen index (FAI) which can be calculated measuring total testosterone and sex hormone binding globuline (SHBG). PCOM is usually determined according to the “International Evidence-based Guideline for PCOS 2018” using endovaginal ultrasound transducers with a frequency bandwidth that includes 8 MHz. The threshold for PCOM is considered to be on either ovary: a follicle number per ovary of >20 and/or an ovarian volume ≥10 ml, ensuring no corpora lutea, cysts or dominant follicles are present. If older ultrasound technology is used, the threshold for PCOM could be an ovarian volume ≥10 ml or a follicle count of >12 on either ovary.

Another method for detecting PCOM is measuring the Anti-Müllerian Hormone (AMH) in a subject. AMH is a glycoprotein hormone whose expression is critical to sex differentiation at a specific time during fetal development. Further, AMH produced by granulosa cells of growing follicles usually correlates with the number of antral follicles within the ovary. Therefore, serum levels of AMH may be a surrogate biomarker for the antral follicle count/number (AFC) determined by transvaginal ultrasound. Some studies have suggested serum AMH as a biochemical marker for PCOM. In some studies, AMH threshold values for PCOM in women with PCOS were proposed (Nicholas et al. 2014; Pigny et al. 2016; Dietz de Loos et al., Fertil Steril, 2021). However, according to the “International evidence-based guideline for the assessment and management of polycystic ovary syndrome 2018” serum AMH levels should not be used as an alternative for the detection of PCOM or to diagnose PCOS.

A further method for detecting PCOS is a 3-item PCOS criteria system (Indran et al. 2018). In this system, it was proposed that diagnosis of PCOS is made, if two out of three items are present: (i) oligomenorrhea (defined as mean menstrual cycle length >35 days); (ii) AMH above threshold; and (iii) hyperandrogenism defined as either testosterone above threshold and/or the presence of hirsutism (mFG score ≥5). Alternatively, AMH was suggested in combination with hyperandrogenism and oligomenorrhea (Sahmay et al., 2014) or in combination with SHBG (Calzada et al., 2019).

Another method for detecting PCOS is to measure other hormones, such as e.g. luteinizing hormone (LH) and follicle-stimulating hormone (FSH). However, the diagnostic utility of the LH:FSH ratio for the diagnosis of PCOS seems low as only a small percentage of women with PCOS have significantly elevated LH:FSH ratios (Cho et al. 2005). Actually, there is a wide range of LH:FSH ratios found in women diagnosed with PCOS (Malini and George 2018).

The necessity to consider the results of multiple diagnostic tests and the results of clinical examination needs specific expertise which makes it quite difficult for less specialized physicians (such as general practitioners) to diagnose PCOS in clinical routine. For example, the determination of PCOM by transvaginal ultrasound requires adequate ultrasound equipment and the subjective analysis of ultrasound images by a physician. Furthermore, the result may also depend on the specific ultrasound device used in the assessment of PCOM. Consequently, the diagnosis of PCOS based on the Rotterdam Criteria always includes at least one subjective, device- and operator-dependent and error-prone measurement.

In order to evaluate biochemical hyperandrogenism, there must be a well-established normal range for the androgen that is measured. Testosterone is the most abundant measured androgen, in its total, bound and free form. The practice of measuring free testosterone has its limitations. Direct measurement of free testosterone using radioimmunoassay is highly inaccurate, and does not reflect the true values. The assays have high intra- and interassay variability. Alternatively, a greater degree of accuracy, particularly for clinical research, will be obtained by measuring total testosterone concentration using extraction and chromatography, or gas (GC-MS) or liquid (LC-MS) chromatography-mass spectrometry. The diagnostic performance of measuring serum testosterone may be enhanced by the concomitant measurement of SHBG, such that the calculation of free T concentration from the total testosterone and SHBG levels only requires solving a second-degree equation (Azziz R, Carmina E, Dewailly D, et al. Task Force on the Phenotype of the Polycystic Ovary Syndrome of The Androgen Excess and PCOS Society. The Androgen Excess and PCOS Society criteria for the polycystic ovary syndrome: the complete task force report. Fertil Steril. 2009; 91(2):456-88). The definition of HA may differ depending on ethnicity. The mFG score of >8 to diagnose hirsutism in women with PCOS may not be appropriate for the diagnosis in all ethnicities. East-Asian women have a lower prevalence of hirsutism compared to Caucasians, and a score of >5 has been proposed for defining hirsutism in Chinese women. There are also indications that the level of androgens in blood differs between ethnicities, where the Japanese population has a lower prevalence of raised androgens and testosterone is only recommended as a complementary factor in the diagnosis of PCOS in this population (Huang Z, Yong E L. Ethnic differences: Is there an Asian phenotype for polycystic ovarian syndrome?Best Pract Res Clin Obstet Gynaecol. 2016; 37:46-55; Kubota T. Update in polycystic ovary syndrome: new criteria of diagnosis and treatment in Japan. Reprod Med Biol. 2013; 12(3):71-77).

Patients suffering from PCOS can be classified in four different phenotypes, named A, B, C or D (Neven A C H, Laven J, Teede H J, Boyle J A. A Summary on Polycystic Ovary Syndrome: Diagnostic Criteria, Prevalence, Clinical Manifestations, and Management According to the Latest International Guidelines. Semin Reprod Med. 2018 January; 36(1):5-12). Phenotype A is characteristic for patients showing hyperandrogenism, ovulatory dysfunction and/or irregular cycles and polycystic ovarian morphology. Phenotype B is characterized by hyperandrogenism, ovulatory dysfunction and/or irregular cycles. Phenotype C is characterized by hyperandrogenism and polycystic ovarian morphology. Phenotype D is characterized by ovulatory dysfunction and/or irregular cycles and polycystic ovarian morphology.

Currently, there is no specific PCOS medication available. Treatment is symptom-oriented and adapted to personal needs. Therapeutic approaches target hyperandrogenism, irregular cycles and/or ovulatory dysfunction and associated metabolic disorders, such as diabetes. The International evidence-based guideline for the assessment and management of polycystic ovary syndrome of 2018 provides information to support clinical decision making and patient management.

Inconsistent diagnostic criteria, variable provider knowledge, and lack of consensus pose specific challenges for the diagnosis and care of women with PCOS. These factors encourage inaccurate diagnosis with both under and over-diagnosis. This unfavorable diagnostic experience exacerbates affected women and limits timely opportunities for intervention to minimize associated comorbidities, especially during the transition from pediatric to adult care (Witchel S F, Teede H J, Pena A S. Curtailing PCOS. Pediatr Res. 2020; 87(2):353-361). Further, timely diagnosis is pivotal to prevent further metabolic complications in affected women, such as e.g. type 2 diabetes mellitus.

In the largest study of PCOS diagnosis experiences, many women reported delayed diagnosis and inadequate information. One-third or more of women reported >2 years (33.6%) and >3 health care professionals (47.1%) before a diagnosis was established. Few were satisfied with their diagnosis experience (35.2%) or with the information they received (15.6%). These gaps in early diagnosis, education, and support are clear opportunities for improving patient experience (Gibson-Helm M, Teede H, Dunaif A, Dokras A. Delayed Diagnosis and a Lack of Information Associated With Dissatisfaction in Women With Polycystic Ovary Syndrome. J Clin Endocrinol Metab. 2017; 102(2):604-612).

An area of particular interest in the diagnosis of PCOS is in women of young age, namely adolescents and young women under the age of 25, when the features of normal pubertal development overlap with adult diagnostic criteria. This makes diagnosis controversial and challenging. Many of the manifestations that are used for diagnosing PCOS may evolve over time and change during the first years after menarche. Normal pubertal physiological changes such as irregular menstrual cycles, acne and PCOM, overlap with adult PCOS diagnostic criteria. In adolescent and young adult women, PCOS is diagnosed when both the OA and HA criteria are fulfilled. The pelvic ultrasound is not recommended to be done in adolescents <8 years from menarche, due to the high incidence of multifollicular ovaries in this life stage (Pena A S, Witchel S F, Hoeger K M, Oberfield S E, Vogiatzi M G, Misso M, Garad R, Dabadghao P, Teede H. Adolescent polycystic ovary syndrome according to the international evidence-based guideline. BMC Med. 2020; 18(1):72). The assessment of an irregular menstrual cycle in adolescents can be difficult. Menstrual cycles are often irregular during adolescence. Immaturity of the hypothalamic-pituitary-ovarian axis during the early years after menarche often results in anovulation and cycles may be somewhat long. However, 90% of cycles will be within the range of 21-45 days, although short cycles of less than 20 days and long cycles of more than 45 days may occur. By the third year after menarche, 60-80% of menstrual cycles are 21-34 days long, as is typical of adults. Young girls and their caretakers (eg, parents or guardians) frequently have difficulty assessing what constitutes normal menstrual cycles or patterns of bleeding. Patients and their caretakers may be unfamiliar with what is normal and patients may not inform their caretakers about menstrual irregularities or missed menses. In addition, a patient is often reluctant to discuss this topic with a caretaker (ACOG Committee Opinion No. 651: Menstruation in Girls and Adolescents: Using the Menstrual Cycle as a Vital Sign. Obstet Gynecol. 2015; 126(6):e143-e146). Hence, in particular for this group of patients, the establishment of a reliable biomarker as an aid for the diagnosis of PCOS or for the identification of patients which are at risk of developing PCOS is of utmost importance. Delay in diagnosing adolescents and young women is often due to unwillingness to diagnose adolescents at risk because of puberty and fear of over- or under-diagnosis. This may lead to long-term complications, such as obesity and insulin resistance, and anxiety or depression. The most recent Guidelines for the diagnosis of PCOS in adolescents and young women have defined oligo-anovulation, irregular menstrual cycle and hyperandrogenism as the criteria to improve diagnostic accuracy in this patient group (Pena A S, Witchel S F, Hoeger K M, et al. Adolescent polycystic ovary syndrome according to the international evidence-based guideline. BMC Med. 2020; 18(1):72). The Guidelines also recommend a re-evaluation of the diagnosis in 3 years intervals and a lifestyle change to minimize symptoms and comorbidities associated with PCOS, such as anxiety and depression.

So far, there is no universal biomarker available, either used alone or in combination with another biomarker, or with the symptoms described above or the hormone levels as described previously, to assess whether a subject has PCOS or is at risk of developing PCOS, and/or to determine response to therapy in a subject with PCOS, and/or to monitor PCOS progression in a subject, and/or to monitor response to treatment in a subject having PCOS.

Therefore, there is an unmet need to establish better diagnostic assays for diagnosing young women and adolescents.

### SUMMARY OF THE INVENTION

In a first aspect, the present invention relates to a method for diagnosing PCOS in a subject, said method comprising the steps of
a) determining the amount or concentration of LTA4H in sample from the subject,
b) determining the amount or concentration of METRNL in a sample from the subject,
c) calculating a score of the amounts or concentrations determined in steps a) and b), and
d) comparing the calculated score with a reference score.
e) diagnosing PCOS in a subject.

Preferably, step e) is based on the results of the comparison step d). Accordingly, step e) may be as follows:
e) diagnosing PCOS in a subject based on the results of the comparison in step d).

In a second aspect, the present invention relates to a method of selecting a patient for therapy of PCOS, comprising:
a) determining the amount or concentration of LTA4H in sample from the subject,
b) determining the amount or concentration of METRNL in a sample from the subject,
c) calculating a score of the amounts or concentrations determined in steps a) and b),
d) comparing the calculated score with a reference score, and
e) selecting the patient for PCOS therapy.

Preferably, step e) is based on the results of the comparison step d). Accordingly, step e) may be as follows:
e) selecting the patient for PCOS therapy based on the results of the comparison in step d).

In a third aspect, the present invention relates to a method for monitoring PCOS progression in a subject having PCOS or for monitoring response to treatment in a subject having PCOS, said method comprising
a) determining the amount or concentration of LTA4H in sample from the subject,
b) determining the amount or concentration of METRNL in a sample from the subject,
c) calculating a score of the amounts or concentrations determined in steps a) and b),
d) comparing the calculated score with a reference score, and
e) monitoring progression in the subject suffering from PCOS or being treated for PCOS.

Preferably, step e) is based on the results of the comparison step d). Accordingly, step e) may be as follows:
e) monitoring progression in the subject suffering from PCOS or being treated for PCOS, based on the results of step d).

In a fourth aspect, the present invention relates to a computer-implemented method for diagnosing PCOS in a subject, said method comprising
(a) receiving at a processing unit
   (a1) a value for the amount or concentration of LTA4H in sample from the subject and
   (a2) a value for the amount or concentration of METRNL in a sample from the subject,
(b) processing the values received in step (a) with the processing unit, wherein said processing comprises
   (b1) calculating a score of the values (a1) and (a2) received in step a),
   (b2) comparing the calculated score with a reference score and
(c) optionally providing the diagnosis via an output device, wherein said diagnosis is based on the results of step b).

In a fifth aspect the present invention relates to the use of LTA4H and METRNL as biomarkers for the diagnosis of PCOS. Further, the invention relates to the use of at least one agent which specifically binds to METRNL and of at least one agent which specifically binds to LTA4H for the diagnosis of PCOS.

In a sixth aspect, the present invention relates to a kit comprising at least one agent that specifically binds to METRNL and at least one agent that specifically binds to LTA4H.

### DESCRIPTION OF THE FIGURES

FIG. 1. ROC curve analysis for the ratio between the two serum biomarkers LTA4H and METRNL for PCOS cases when all phenotypes were combined (phenotypes A-D). The ratio was performed between the biomarker concentrations obtained by ELISA immunoassays.

FIG. 2. Values of the ratio between the two serum biomarkers LTA4H and METRNL in controls versus PCOS cases when all phenotypes were combined (phenotypes A-D). The ratio was performed between the biomarker concentrations obtained by ELISA immunoassays.

FIG. 3. ROC curve analysis for the ratio between the two serum biomarkers LTA4H and METRNL for PCOS phenotypes A-D when compared to controls. The ratio was performed between the biomarker concentrations obtained by ELISA immunoassays.

FIG. 4. Values of the ratio between the two serum biomarkers LTA4H and METRNL in healthy controls and in PCOS phenotypes A-D. The ratio was performed between the biomarker concentrations obtained by ELISA immunoassays.

FIG. 5. ROC curve analysis for the ratio between the two serum biomarkers LTA4H and METRNL for young PCOS cases (age ≤25) when all phenotypes A-D were combined. The ratio was performed between the biomarker concentrations obtained by ELISA immunoassays.

FIG. 6. Values of the ratio between the two serum biomarkers LTA4H and METRNL in young controls versus young PCOS cases (age ≤25) combined phenotypes A-D. The ratio was performed between the biomarker concentrations obtained by ELISA immunoassays.

FIG. 7. ROC curve analysis for the ratio between the two serum biomarkers LTA4H and METRNL for young PCOS cases (age 25) separated by phenotypes A-D versus young healthy controls (age ≤25). The ratio was performed between the biomarker concentrations obtained by ELISA immunoassays.

FIG. 8. Values of the ratio between the two serum biomarkers LTA4H and METRNL in young healthy controls (age ≤25) and in PCOS phenotypes A-D (age ≤25). The ratio was performed between the biomarker concentrations obtained by ELISA immunoassays.

FIG. 9. ROC curve analysis for the ratio between the two serum biomarkers LTA4H and METRNL for PCOS cases when all phenotypes were combined (phenotypes A-D). The ratio was performed between the biomarker concentrations obtained by ELISA immunoassays.

FIG. 10. Values of the ratio between the two serum biomarkers LTA4H and METRNL in controls versus PCOS cases when all phenotypes were combined (phenotypes A-D). The ratio was performed between the biomarker concentrations obtained by ELISA immunoassays.

FIG. 11. ROC curve analysis for the ratio between the two serum biomarkers LTA4H and METRNL for PCOS phenotypes A-D when compared to controls. The ratio was performed between the biomarker concentrations obtained by ELISA immunoassays.

FIG. 12. Values of the ratio between the two serum biomarkers LTA4H and METRNL in healthy controls and in PCOS phenotypes A-D. The ratio was performed between the biomarker concentrations obtained by ELISA immunoassays.

FIG. 13. ROC curve analysis for the ratio between the two serum biomarkers LTA4H and METRNL for different PCOS age groups (15-20, 20-25, 25-45) when compared to controls. The ratio was performed between the biomarker concentrations obtained by ELISA immunoassays.

FIG. 14. Values of the ratio between the two serum biomarkers LTA4H and METRNL in healthy controls and in PCOS age groups (15-20, 20-25, 25-45). The ratio was performed between the biomarker concentrations obtained by ELISA immunoassays.

FIG. 15. ROC curve analysis for the ratio between the two serum biomarkers LTA4H and METRNL for young PCOS cases (age 15-25) when all phenotypes A-D were combined. The ratio was performed between the biomarker concentrations obtained by ELISA immunoassays.

FIG. 16. Values of the ratio between the two serum biomarkers LTA4H and METRNL in young healthy controls versus young PCOS cases (age 15-25), combined phenotypes A-D. The ratio was performed between the biomarker concentrations obtained by ELISA immunoassays.

FIG. 17. ROC curve analysis for the ratio between the two serum biomarkers LTA4H and METRNL for young PCOS cases (age 15-25) separated by phenotypes A-D versus young healthy controls (age 15-25). The ratio was performed between the biomarker concentrations obtained by ELISA immunoassays.

FIG. 18. Values of the ratio between the two serum biomarkers LTA4H and METRNL in young healthy controls (age 15-25) and in PCOS phenotypes A-D (age 15-25). The ratio was performed between the biomarker concentrations obtained by ELISA immunoassays.

### DETAILED DESCRIPTION OF THE INVENTION

The methods as referred to in accordance with the first, the second or the third aspects of the present invention include methods which essentially consist of the aforementioned steps or methods which include further steps. Moreover, the methods of the present invention, preferably, are ex vivo, and more preferably in vitro methods. Moreover, the methods according to the first, the second or the third aspects of the present invention may comprise steps in addition to those explicitly mentioned above. For example, further steps may relate to the determination of further markers and/or to sample pre-treatments or evaluation of the results obtained by the method. The methods may be carried out manually or assisted by automation. Preferably, step (a), (b), (c), (d) and/or (c) may in total or in part be assisted by automation, e.g., by a suitable robotic and sensory equipment for the determination in step (a) and (b) or a computer-implemented calculation in step (c) or a computer-implemented comparison in step (d). Thus, the methods of the present invention may be computer-implemented.

Leukotriene A4 Hydrolase (LTA4H) is part of the 5-lipoxygenase (5-LO) pathway which converts arachidonic acid (AA) into pro-inflammatory leukotrienes and anti-inflammatory lipoxins. LTA4H is widely expressed in various organs, tissues and individual cell types (Haeggstram J Z. Leukotriene A4 hydrolase and the committed step in leukotriene B4 biosynthesis. Clin Rev Allergy Immunol. 1999 Spring-Summer; 17(1-2):111-31). According to the Human Protein Atlas a medium expression of LTA4H is detected in normal ovary tissues (http://www.proteinatlas.org). The patterns of expression of LTA4H have been investigated in human ovarian tissues, revealing weak and moderate expression in the preovulatory follicles on the granulosa and theca interna cells, respectively. After ovulation, the intensity of LTA4H on large luteal cells increased and was highest in the midluteal phase. High expression was also observed in the corpus luteum of early pregnancy (Hattori N, Fujiwara H, Maeda M, et al. Human large luteal cells in the menstrual cycle and early pregnancy express leukotriene A4 hydrolase. Mol Hum Reprod. 1998 August; 4(8):803-10). LTA4H is a unique bi-functional zinc metalloenzyme with both epoxide hydrolase (intracellularly) and aminopeptidase (extracellularly) activities. As epoxide hydrolase, LTA4H catalyzes the final and committed step in leukotriene B4 (LTB4) biosynthesis, an eicosanoid that has potent chemoattractant and pro-inflammatory properties (Serhan C N. Resolution phase of inflammation: novel endogenous anti-inflammatory and proresolving lipid mediators and pathways. Annual review of immunology. 2007; 25: 101-137). Leukotrienes are a family of cicosanoids that function as potent chemical mediators in a variety of allergic and inflammatory reactions. Emerging data suggest that leukotrienes can have an important role in carcinogenesis. LTB4 levels are increased in several human cancers, including skin, lung, colon and prostate cancers (Dreyling K W, Hoppe U, Peskar B A, et al Leukotriene synthesis by human gastrointestinal tissues. Biochim Biophys Acta. 1986 Sep. 12; 878(2):184-93; Chen X, Wang S, Wu N, Yang C S. Leukotriene A4 hydrolase as a target for cancer prevention and therapy. Curr Cancer Drug Targets. 2004 May; 4(3):267-83; Wang Q, He Z, Zhang J, et al. Overexpression of endoplasmic reticulum molecular chaperone GRP94 and GRP78 in human lung cancer tissues and its significance. Cancer Detect Prev. 2005; 29(6):544-51; Larré S, Tran N, Fan C, et al. PGE2 and LTB4 tissue levels in benign and cancerous prostates. Prostaglandins Other Lipid Mediat. 2008 December; 87(1-4):14-9; Oi N, Yamamoto H, Langfald A, et al. LTA4H regulates cell cycle and skin carcinogenesis. Carcinogenesis. 2017 Jul. 1; 38(7):728-737). The expression of LTB4 receptors is increased in human pancreatic cancer (Hennig R, Ding X Z, Tong W G, et al. 5-Lipoxygenase and leukotriene B(4) receptor are expressed in human pancreatic cancers but not in pancreatic ducts in normal tissue. Am J Pathol. 2002 August; 161(2):421-8). LTB4 expression is also increased in HRAS-v12-transformed cells and the receptor BLT2 is required for Ras-induced transformation in vivo (Yoo M H, Song H, Woo C H, et al. Role of the BLT2, a leukotriene B4 receptor, in Ras transformation. Oncogene. 2004 Dec. 9; 23(57):9259-68). Inhibition of LTB4 synthesis by treatment with an LTA4H inhibitor, bestatin, reduced tumorigenesis in in vivo models of esophageal adenocarcinoma and of colorectal cancer (Chen X, Li N, Wang S, et al. Leukotriene A4 hydrolase in rat and human esophageal adenocarcinomas and inhibitory effects of bestatin. J Natl Cancer Inst. 2003 Jul. 16; 95(14):1053-61; Zhao S, Yao K, Li D, et al. Inhibition of LTA4H by bestatin in human and mouse colorectal cancer. EBioMedicine. 2019 June; 44:361-374). Furthermore, topical application of LTB4 to the skin led not only to inflammation but also to substantial hyperplasia in the epidermis (Bauer R W, van der Kerhof P C, de Grood R M. Epidermal hyperproliferation following the induction of microabscesses by leukotriene B4. Br J Dermatol 1986; 114:409-12; Ruzicka T, Burg G. Effects of chronic intracutaneous administration of arachidonic acid and its metabolites. Induction of leukocytoclastic vasculitis by leukotriene B4 and 12-hydroxyeicosatetraenoic acid and its prevention by prostaglandin E2. J Invest Dermatol 1987; 88:120-3). The expression of 5-lipoxygenase, of the hypoxia marker HIF1α and of the macrophage marker CD68, was positively correlating in ovarian cancer tissues. Furthermore, hypoxic ovarian cancer cell lines showed increased production of LTA4H and 5-LOX metabolites, responsible for enhancing the infiltration of tumor-associated macrophages (Wen Z, Liu H, Li M, et al. Increased metabolites of 5-lipoxygenase from hypoxic ovarian cancer cells promote tumor-associated macrophage infiltration. Oncogene. 2015 Mar. 5; 34(10):1241-52). LTA4H may also counteract inflammation by its aminopeptidase activity, which inactivates by cleavage the tripeptide Pro-Gly-Pro (PGP) (Stsiapanava A, Olsson U, Wan M, et al. Binding of Pro-Gly-Pro at the active site of leukotriene A4 hydrolase/aminopeptidase and development of an epoxide hydrolase selective inhibitor. Proc Natl Acad Sci USA. 2014 Mar. 18; 111(11):4227-32). PGP is a biomarker for chronic obstructive pulmonary disease (COPD) and promotes neutrophil accumulation (Snelgrove R J, Jackson P L, Hardison M T, et al. A critical role for LTA4H in limiting chronic pulmonary neutrophilic inflammation. Science. 2010 Oct. 1; 330(6000):90-4). Serum LTA4H was recently suggested as a potential biomarker to predict the efficiency of allergen immunotherapy for the treatment of allergic rhinitis (Ma T T, Cao M D, Yu R L, et al. Leukotriene A4 Hydrolase Is a Candidate Predictive Biomarker for Successful Allergen Immunotherapy. Front Immunol. 2020 Nov. 24; 11:559746). Higher expression of LTA4H, due to a LTA4H genetic variant, was associated with increased survival upon glucocorticoid therapy in tuberculous meningitis patients with moderate disease (Whitworth L, Coxon J, van Laarhoven A, et al. A Bayesian analysis of the association between Leukotriene A4 Hydrolase genotype and survival in tuberculous meningitis. Elife. 2021 Jan. 8; 10:e61722).

Meteorin-like protein (METRNL) is a hormone (28 KDa secreted protein) that is induced after exercise and cold exposure in the skeletal muscle and adipose tissue, respectively. Increased expression of METRNL in the circulation or in adipose tissue resulted in ‘browning’ of the white adipose tissue (WAT). Intraperitoneal injection with Metrnl-Fe protein in mice for 7 days induced significant body weight reduction, increased 02 consumption and glucose tolerance. Metrnl did not show direct effect on the thermogenesis of white adipocytes in vitro, which indicated the involvement of a non-adipose cell type in the induction of beige fat. Metrnl seemed to rather stimulate several immune cell subtypes to enter the adipose tissue and to activate their prothermogenic actions. METRNL-treated mice had increased macrophage and eosinophils numbers in WAT and increased expression of genes that are associated with alternative macrophage activation (Rao R R, Long J Z, White J P, et al. Meteorin-like is a hormone that regulates immune-adipose interactions to increase beige fat thermogenesis. Cell. 2014 Jun. 5; 157(6):1279-1291). METRNL has been associated with innate and possibly acquired immunity. High expression of METRNL was identified in activated monocytes (M2-polarized macrophages), skin and mucosal tissues. In the skin, METRNL is expressed by resting fibroblasts and IFNγ-treated keratinocytes. Over-expression of METRNL was described in several human skin diseases including psoriasis. METRNL is also up-regulated in synovial membranes of human rheumatoid arthritis (Ushach I, Burkhardt A M, Martinez C, et al. METEORIN-LIKE is a cytokine associated with barrier tissues and alternatively activated macrophages. Clin Immunol. 2015 February; 156(2):119-27). Recently, Baht and colleagues described a role for METRNL in coordinating skeletal muscle repair through macrophage accretion and phenotypical switch. The results suggested that in response to local injury METRNL would be secreted predominantly from macrophages. Furthermore, METRNL promoted an anti-inflammatory function through a STAT3-dependent auto-/paracrine mechanism inducing insulin-like growth factor 1 (IGF-1), which activated muscle progenitors to help myogenesis.

Finally, METRNL has been shown to be a critical regulator of muscle regeneration acting directly on immune cells to promote an anti-inflammatory/pro-regenerative environment and myogenesis (Baht G S, Bareja A, Lee D E, et al. Meteorin-like facilitates skeletal muscle repair through a Stat3/IGF-1 mechanism. Nat Metab. 2020 March; 2(3):278-289. Erratum in: Nat Metab. 2020 August; 2(8):794). Interestingly, METRNL has been suggested to act as a neurotrophic factor with therapeutic potential in neural development. METRNL is indeed able to cross the blood brain barrier (BBB) and an increasing blood-brain barrier dysfunction caused increasing cerebrospinal fluid METRNL concentrations (Berghoff M, Höpfinger A, Rajendran R, et al. Evidence of a Muscle-Brain Axis by Quantification of the Neurotrophic Myokine METRNL (Meteorin-Like Protein) in Human Cerebrospinal Fluid and Serum. Journal of Clinical Medicine. 2021; 10(15):3271).

Serum METRNL levels have been studied in association with type 2 diabetes (T2DM) producing conflicting results (Lee J H, Kang Y E, Kim J M, et al. Serum Meteorin-like protein levels decreased in patients newly diagnosed with type 2 diabetes. Diabetes Res Clin Pract. 2018 January; 135:7-10; Chung H S, Hwang S Y, Choi J H, et al. Implications of circulating Meteorin-like (Metrnl) level in human subjects with type 2 diabetes. Diabetes Res Clin Pract. 2018 February; 136:100-107; Wang K, Li F, et al. Serum Levels of Meteorin-Like (Metrnl) Are Increased in Patients with Newly Diagnosed Type 2 Diabetes Mellitus and Are Associated with Insulin Resistance. Med Sci Monit. 2019 Mar. 31; 25:2337-2343; El-Ashmawy H M, Selim F O, Hosny T A M, Almassry H N. Association of low serum Meteorin like (Metrnl) concentrations with worsening of glucose tolerance, impaired endothelial function and atherosclerosis. Diabetes Res Clin Pract. 2019 April; 150:57-63; Wang C, Pan Y, Song J, et al. Serum Metrnl Level is Correlated with Insulin Resistance, But Not with β-Cell Function in Type 2 Diabetics. Med Sci Monit. 2019 Nov. 25; 25:8968-8974; Ferns G A, Fekri K, Shahini Shams Abadi M, et al. A meta-analysis of the relationship between scrums metrnl-like protein/subfatin and risk of type 2 diabetes mellitus and coronary artery disease. Arch Physiol Biochem. 2021 May 5:1-7; Lappas M. Maternal obesity and gestational diabetes decrease Metrnl concentrations in cord plasma. J Matern Fetal Neonatal Med. 2021 September; 34(18):2991-2995). Patients with T2DM and coronary artery disease (CAD) showed lower serum levels of METRNL compared to the control group. Additionally, METRNL illustrated a negative correlation with IL-6 and TNF-α in both CAD patients and also with BMI, insulin resistance, IL-6 and TNF-α in T2DM patients (Dadmanesh M, Aghajani H, Fadaei R, Ghorban K. Lower serum levels of Meteorin-like/Subfatin in patients with coronary artery disease and type 2 diabetes mellitus are negatively associated with insulin resistance and inflammatory cytokines. PLoS One. 2018 Sep. 13; 13(9):e0204180). Furthermore, a case-control study for CAD patients showed significant associations of serum METRNL with the presence and severity of CAD (Liu Z X, Ji H H, Yao M P, et al. Serum Metrnl is associated with the presence and severity of coronary artery disease. J Cell Mol Med. 2019 January; 23(1):271-280). Obese patients undergoing bariatric surgery showed decreased circulating levels of METRLN and improvement in glucose and lipid homeostasis compared to normal-weight controls (Pellitero S, Piquer-Garcia I, Ferrer-Curriu G, et al. Opposite changes in meteorin-like and oncostatin m levels are associated with metabolic improvements after bariatric surgery. Int J Obes (Lond). 2018 April; 42(4):919-922). Recently two studies have investigated circulating levels of METRNL in PCOS patients compared to controls. Fouani et al. study was conducted on a cohort of PCOS-recurrent pregnancy loss (PCOS-RPL, n=60) and infertile PCOS (n=60) patients and 60 healthy controls. Women's age was from 20 to 40 years (average of controls' age: 30.02±4.60; average of PCOS cases' age: 29.88±4.22). The authors found lower serum METRNL levels in PCOS patients when compared to controls. Moreover, serum METRNL correlated with BMI, adiponectin, and homocysteine in controls, and inversely correlated with FBG, fasting insulin, and HOMA-IR in PCOS group and subgroups. Besides, it inversely correlated with hs-CRP in control, and PCOS group and subgroups (Fouani F Z, Fadaei R, Moradi N, et al. Circulating levels of Meteorin-like protein in polycystic ovary syndrome: A case-control study. PLoS One. 2020 Apr. 24; 15(4):e0231943). Deniz et al. measured METRNL (subfatin) and asprosin levels in plasma samples from 30 PCOS cases and 30 healthy controls (average of controls' age: 28.22±2.6; average of PCOS cases' age: 27.14±3.21). While asprosin levels were significantly higher in women with PCOS compared to healthy controls, METRNL levels were significantly lower compared to controls, in agreement with the results shown by Fouani et al. Both asprosin and METRNL levels showed significant correlation with HOMA-IR in the PCOS subgroup (Deniz R, Yavuzkir S, Ugur K, et al. Subfatin and asprosin, two new metabolic players of polycystic ovary syndrome. J Obstet Gynaccol. 2021 February; 41(2):279-284. doi: 10.1080/01443615.2020.1758926).

In Inflammatory Bowel Disease (IBD) patients, serum levels of METRNL were decreased and a negative correlation was identified with TNF-α, IL-6 and BMI levels (Gholamrezayi A, Mohamadinarab M, Rahbarinejad P, et al. Characterization of the serum levels of Meteorin-like in patients with inflammatory bowel disease and its association with inflammatory cytokines. Lipids Health Dis. 2020 Oct. 30; 19(1):230). In line with other studies on metabolic and inflammatory diseases, in osteoarthritic patients, compared to non-osteoarthritic subjects, serum METRNL was lower while synovial fluid METRNL was higher (Sobieh B H, Kassem D H, Zakaria Z M, El-Mesallamy H O. Potential emerging roles of the novel adipokines adipolin/CTRP12 and meteorin-like/METRNL in obesity-osteoarthritis interplay. Cytokine. 2021 February; 138:155368).

There is an unmet medical need for an accurate test for the reliable diagnosis of PCOS. Measurements of the ratio between LTA4H and METRNL in a sample have the advantage of a reliable biological fluid—based test that identifies women suffering from PCOS that is currently not possible. This method can also be reliably used in adolescent subjects and young women under the age of 25 years, in particular under the age of 20 years, in particular 15 to under the age of 25 years, in particular 15 to under the age of 20 years, for the diagnosis of PCOS. Diagnosis of PCOS in adolescent patients is difficult for the reasons described above and hence, for the first time, we provide an accurate test for the diagnosis of PCOS in the adolescent and young women population. In addition, measurements of the ratio between LTA4H and METRNL in a sample have the advantage to identify if the patient responds to therapy. An additional advantage of measurements of the ratio between LTA4H and METRNL in a sample of a patient is to monitor the progression of PCOS. Furthermore, we enclose a computer-implemented method for assessing a subject suffering from PCOS by measuring the ratio between LTA4H and METRNL levels in a sample and, optionally, with further criteria such as a value for oligo-anovulation and/or irregular cycles, hyperandrogenism and/or polycystic ovarian morphology or with further biomarkers or hormones, for assessing said subject based on the comparison and/or the calculation of the data described above.

As described above, patients suffering from PCOS may show two types of characteristics—reproductive or metabolic type. The metabolic type of PCOS include obesity, insulin resistance, metabolic syndrome, pre-diabetes, type-2 diabetes, nonalcoholic fatty liver disease (NAFLD), and cardiovascular factors. The term “phenotypical” can be used instead of “reproductive”. The term “reproductive” (or “phenotypical”) describes any feature of the phenotype of a female known to be indicative of PCOS. For example, these reproductive characteristics comprise polycystic ovarian morphology (PCOM) and/or clinical hyperandrogenism, such as acne, seborrhea, alopecia, and/or hirsutism. Preferably, these reproductive characteristics comprise polycystic ovarian morphology (PCOM) and/or clinical hyperandrogenism, more preferably acne, seborrhea, alopecia, deepening of voice and/or hirsutism. These reproductive characteristics of clinical hyperandrogenism may be simply diagnosed by asking the female or are apparent after a short physical examination of the female's body. Usually, a reference population does not show any or not more than one of these phenotypical characteristics known to be indicative of PCOS.

In embodiments, PCOS is assessed from the group consisting of metabolic or phenotypical PCOS. In further embodiments, PCOS is assessed from the group consisting of phenotype A, phenotype B, phenotype C and phenotype D PCOS.

In embodiments, the methods according to the present invention are an in vitro methods.

The term “diagnosing” as used herein, preferably, means assessing whether a subject as referred to in accordance with the method of the present invention suffers from PCOS or not. Preferably, the expression “diagnosing PCOS” as used herein shall be understood as “aiding” or “assisting” in the diagnosis of PCOS. For example, a physician may be assisted in the diagnosis of PCOS by additional information and/or devices. Thus, the actual diagnosis might be carried out by a physician.

As will be understood by those skilled in the art, the diagnosis of the present invention is usually not intended to be correct for 100% of the subjects to be tested. The term “diagnosing”, preferably, requires that a correct diagnosis can be made for a statistically significant portion of subjects. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98%, or at least 99%. The p-values are, preferably, 0.4, 0.1, 0.05, 0.01, 0.005, or 0.0001.

The term “sample” refers to a sample of a body fluid, to a sample of separated cells or to a sample from a tissue or an organ. Samples of body fluids can be obtained by well-known techniques and include, preferably, samples of blood, plasma, serum, capillary blood, interstitial fluid, peritoneal fluid, menstrual fluid, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from those cells, tissues or organs which express or produce the peptides referred to herein.

In some embodiments of the method of the present invention, the sample is blood sample (i.e. a whole blood sample), a serum sample, or a plasma sample.

The term “subject” as referred to herein is, preferably, a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the subject is a human. The subject can be male or female. The terms “patient” and “subject” are used interchangeably herein. In particular embodiments, the patient is a female human patient of less than 25 years old. In particular embodiments, the patient is a female human patient of less than 20 years old. In particular embodiments, the patient is a female human patient of 15 to less than 25 years old. In particular embodiments, the patient is a female human patient of 15 to less than 20 years old. In particular embodiments, the patient is a female human patient of less than 25 years old and three years after menarche. In particular embodiments, the patient is a female human patient of less than 20 years old and three years after menarche. In particular embodiments, the patient is a female human patient of 15 to less than 25 years old and three years after menarche. In particular embodiments, the patient is a female human patient of 15 to less than 20 years old and three years after menarche.

According to step a) of the methods of the present invention, the amount or concentration of LTA4H is determined, i.e. measured, in a sample from the subject. According to step b), the amount or concentration of METRNL is determined in a sample, from the subject. It is to be understood that steps a) and b) can be carried out in any order. Further, the steps may be carried out simultaneously.

In embodiments, the amounts or concentrations of LTA4H and METRNL are determined using antibodies, in particular using monoclonal antibodies. In embodiments, steps a) and b) of determining the amounts or concentrations of LTA4H and METRNL in a sample of the patient comprises performing an immunoassay. In embodiments, the immunoassay is performed either in a direct or indirect format. In embodiments, such immunoassay is selected from the group consisting of enzyme linked immunosorbent assay (ELISA), enzyme immunoassay (EIA), radioimmunoassay (RIA), or immuno assay based on detection of luminescence, fluorescence, chemiluminescence or electrochemiluminescence.

In particular embodiments, step a) of determining the amount or concentration of LTA4H in a sample of the patient comprises the steps of:
i) incubating the sample of the patient with one or more antibodies specifically binding to LTA4H, thereby generating a complex between the antibody and LTA4H, and
ii) quantifying the complex formed in step i), thereby quantifying the amount or concentration of LTA4H in the sample of the patient.

In particular embodiments, in step i) the sample is incubated with two antibodies, specifically binding to LTA4H. As obvious to the person skilled in the art, the sample can be contacted with the first and the second antibody in any desired order, e.g. first antibody first and then the second antibody or second antibody first and then the first antibody, or simultaneously, for a time and under conditions sufficient to form a first anti-LTA4H antibody/LTA4H/second anti-LTA4H antibody complex. As the person skilled in the art will readily appreciate it is nothing but routine experimentation to establish the time and conditions that are appropriate or that are sufficient for the formation of a complex either between the specific anti-LTA4H antibody and the LTA4H antigen/analyte (=anti-LTA4H complex) or the formation of the secondary, or sandwich complex comprising the first antibody to LTA4H, LTA4H (the analyte) and the second anti-LTA4H antibody (=first anti-LTA4H antibody/LTA4H/second anti-LTA4H antibody complex).

The detection of the anti-LTA4H antibody/LTA4H complex can be performed by any appropriate means. The detection of the first anti-LTA4H antibody/LTA4H/second anti-LTA4H antibody complex can be performed by any appropriate means. The person skilled in the art is absolutely familiar with such means/methods.

In certain embodiments, a sandwich will be formed comprising a first antibody to LTA4H, LTA4H (analyte) and the second antibody to LTA4H, wherein the second antibody is detectably labeled.

In one embodiment, a sandwich will be formed comprising a first antibody to LTA4H, LTA4H (analyte) and the second antibody to LTA4H, wherein the second antibody is detectably labeled and wherein the first anti-LTA4H antibody is capable of binding to a solid phase or is bound to a solid phase.

In a preferred embodiment, determining the amount or concentration of METRNL in a sample from the subject comprises contacting the sample with an antibody, or antigen-binding fragment thereof, which specifically detects METRNL. Preferably, the antibody, or antigen-binding fragment thereof, which specifically detects METRNL specifically binds to an epitope of METRNL. The formed complex between the antibody (or fragment) and the biomarker shall be proportional to the amount or concentration of the METRNL.

Preferably, the determination of the amount or concentration of LTA4H comprises contacting the sample with an antibody, or antigen-binding fragment thereof, which specifically detects LTA4H. Preferably, the antibody, or antigen-binding fragment thereof, which specifically detects LTA4H specifically binds to an epitope of LTA4H. The formed complex between the antibody (or fragment) and the biomarker shall be proportional to the amount or concentration of the LTA4H.

The term “antibody” is known in the art. As used herein, the term refers to any immunoglobulin (Ig) molecule comprised of four polypeptide chains, two heavy (H) chains and two light (L) chains. As used herein, the term “antibody” also includes an antigen-binding fragment of the antibody. As used herein, an antigen-binding fragment of an antibody shall be capable of specifically binding to the antigen. Thus, antigen binding fragments of antibodies are fragments retaining the ability of the (full-length) antibody to specifically bind to the antigen (such as METRNL or LTA4H). Antibody fragments preferably comprise a portion of a full length antibody, preferably the variable domain thereof, or at least the antigen binding site thereof. In an embodiment, the antigen-binding fragment is selected from the group consisting of a Fab fragment, a Fab′ fragment, a Facb fragment, a F(ab′)2 fragment, a scFv fragment, an a Fv fragment. For example, the antigen-binding fragment is a F(ab′)2 fragment. How to produce antigen-binding fragments is well known in the art. For example, the fragments can be produced by enzymatic cleavage of an antibody of the present invention. In addition, the fragments can be generated by synthetic or recombinant techniques. Fab fragments are preferably generated by papain digestion of an antibody, Fab′ fragments by pepsin digestion and partial reduction, F(ab′)2 fragments by pepsin digestion), and facb fragments by plasmin digestion. Fv or scFv fragments are preferably produced by molecular biology techniques.

The antibody in accordance with the methods of the present invention can be a polyclonal or monoclonal antibody. In a preferred embodiment, the antibody is a monoclonal antibody. The term “monoclonal antibody” is well known in the art. As used herein, the term preferably refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. A monoclonal antibody of the present invention can be made by the well-known hybridoma method described by Kohler and Milstein, Nature, 256:495 (1975), or can be made by recombinant DNA methods. In some embodiments, the monoclonal antibody is selected from a group consisting of a sheep monoclonal antibody, a mouse monoclonal antibody, a rabbit monoclonal antibody, a goat monoclonal antibody, a horse monoclonal antibody, a chicken monoclonal antibody. In some embodiments, the monoclonal antibody is a mouse monoclonal antibody.

The antibodies (or fragments) that are used in steps a) and b) of the methods of the present invention can be used in a sandwich assay as capture antibody in combination with at least one other antibody binding to a different epitope.

The antibodies can be used in sandwich assays. Sandwich assays are among the most useful and commonly used assays encompassing a number of variations of the sandwich assay technique. For example, in a typical assay, an unlabeled (capture) binding agent is immobilized or can be immobilized on a solid substrate, and the sample to be tested is brought into contact with the capture binding agent. After a suitable period of incubation, for a period of time sufficient to allow formation of a binding agent-biomarker complex, a second (detection) binding agent labeled with a reporter molecule capable of producing a detectable signal is then added and incubated, allowing time sufficient for the formation of another complex of binding agent-biomarker-labeled binding agent. Any unreacted material may be washed away, and the presence of the biomarker is determined by observation of a signal produced by the reporter molecule bound to the detection binding agent. The results may either be qualitative, by simple observation of a visible signal, or may be quantitated by comparison with a control sample containing e.g. known amounts of the biomarker to be determined (as standard or calibrator as described elsewhere herein).

The incubation steps of a typical sandwich assay can be varied as required and appropriate. Such variations include for example simultaneous incubations, in which two or more binding agents and biomarkers are co-incubated. For example, both the sample to be analyzed and a labeled binding agent are added simultaneously to an immobilized capture binding agent. It is also possible to first incubate the sample to be analyzed and a labeled binding agent and to thereafter add an antibody bound to a solid phase or capable of binding to a solid phase.

The formed complex between a specific binding agent and the biomarker shall be proportional to the amount or concentration of the biomarker present in the sample. It will be understood that the specificity and/or sensitivity of the binding agent to be applied defines the degree of proportion of at least one marker comprised in the sample, which is capable of being specifically bound. Further details, on how the measurement can be carried out, are also found elsewhere herein. The amount or concentration of formed complex shall be transformed into an amount or concentration of the biomarker reflecting the amount or concentration indeed present in the sample.

The term “amount” as used herein encompasses the absolute amount of LTA4H or of METRNL, the relative amount or concentration of the said LTA4H or METRNL, as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters, which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations. According to preferred embodiments of the subject invention, the determination of an “amount” is performed by the disclosed system, whereby a computing device determines the “amount” based on contacting and measuring steps performed by one or more analyzer units of said system.

In step c) of the methods according to the first, or the second or the third aspects of the present invention, a score of the amounts or concentrations determined in steps a) and b), i.e. the amount or concentration of LTA4H and the amount or concentration of METRNL is calculated.

The term “calculating” as used herein refers to assessing a score, which is based on the amount or concentration of LTA4H and the amount or concentration of METRNL determined in the sample(s) of the subject. For example, it is envisaged to calculate a score based on the amount or concentration of LTA4H and the amount or concentration of METRNL, i.e. a single score, and to compare this score to a reference score. The calculated score combines information on the amount or concentration of LTA4H and the amount or concentration of METRNL. Moreover, the biomarkers may be weighted in the score in accordance with their contribution to the establishment of the diagnosis. The score can be regarded as a classifier parameter for diagnosing PCOS. In particular, the score shall enable the diagnosis of PCOS based on the comparison with a reference score. The reference score is preferably a value, in particular a cut-off value, which allows for differentiating between a subject who suffers from PCOS and a subject who does not suffer from PCOS.

Preferably, the score is a ratio, i.e. a ratio of the amount or concentration of LTA4H and the amount or concentration of METRNL. Thus, the ratio calculated in step c) according to the first, or the second, or the third aspects of the present invention, is compared to a reference ratio. In an embodiment, the ratio is the ratio of the amount or concentration of LTA4H to the amount or concentration of METRNL. In an alternative embodiment, the ratio is the ratio of the amount or concentration of METRNL to the amount or concentration of LTA4H.

In step d) according to the first, or the second, or the third aspects of the method of the present invention, the score calculated in step c) shall be compared with a reference score. For example, a calculated ratio shall be compared to a reference ratio.

The term “comparing” as used herein encompasses comparing the score calculated for a sample from a test subject, which a suitable reference source specified elsewhere in this description. The comparison is, preferably, assisted by automation. For example, a suitable computer program comprising algorithms for the comparison of subject's calculated score and the reference score may be used. Such computer programs and algorithms are well known in the art. Notwithstanding the above, a comparison can also be carried out manually. The computer program may further evaluate the result of the comparison, i.e. automatically provides the desired assessment in a suitable output format, i.e. the diagnostic result. The said diagnostic result may, preferably, serve as an aid for establishing the final diagnosis of PCOS by, e.g., a medical practitioner.

The calculation step and/or the comparison step may be carried out by using a computer comprising a processing unit.

Based on the comparison of the calculated score with the reference score, it shall be possible to assess whether the test subject suffers from PCOS, or not. For example, a result of a comparison may be given as raw data, and in some cases as an indicator in the form of a word, phrase, symbol, or numerical value which may be indicative of a particular diagnosis. Therefore, the reference score to be chosen so that either a difference or an identity of the calculated score to the calculated score allows for identifying those test subjects which belong into the group of subjects which suffer from PCOS, or not. The method allows either excluding (rule-out) or identifying (rule-in) a subject who is suffering from PCOS. Differences in the score, i.e. increases or decreases, as used herein, preferably, are differences which are statistically significant. The method may also use other diagnostic criteria such as oligo-anovulation or hyperandrogenism to assess whether a patient suffers from PCOS. (page 26, third paragraph)

Preferably, the reference score, such as the reference ratio, shall allow for differentiating whether a subject suffers from PCOS, or not. Preferably, the diagnosis is made by assessing whether the score of the test subject is above or below the reference score. It is not necessary to provide an exact reference score. A relevant reference score can be obtained by correlating the sensitivity and specificity and the sensitivity/specificity for any score. A reference score resulting in a high sensitivity results in a lower specificity and vice versa.

In some embodiments, the reference score is derived from a sample from a subject (or from samples group of subjects) known to suffer from PCOS.

In some embodiments, the reference score is derived from a sample from a subject (or from samples group of subjects) known not to suffer from PCOS.

As set forth above, the score calculated in step c) according to the first, or the second, or the third aspects of the present invention, may be a ratio. In an embodiment, the ratio is the ratio of the amount or concentration of LTA4H to the amount or concentration of METRNL. In this case, a ratio (i.e. a calculated ratio) which is larger than the reference ratio is indicative for a subject who suffers from PCOS. A ratio which is lower than the reference ratio is indicative for a subject who does not suffer from PCOS. In an alternative embodiment, the calculated ratio is the ratio of the amount or concentration of METRNL to the amount or concentration of LTA4H. In this case, a ratio (i.e. a calculated ratio) which is lower than the reference ratio is indicative for a subject who suffers from PCOS. A ratio which is larger than the reference ratio is indicative for a subject who does not suffer from PCOS.

In a preferred embodiment, the methods of the present invention further comprises the step of recommending a suitable therapy, if PCOS has been diagnosed. Alternatively, the methods further comprises the step of initiating a suitable therapy, if PCOS has been diagnosed.

The term “recommending” as used herein means establishing a proposal for a therapy which could be applied to the subject. However, it is to be understood that applying the actual therapy whatsoever is not comprised by the term. The therapy to be recommended depends on the out-come of the diagnosis provided by the method of the present invention. The recommendation step referred to above can also, preferably, be automated. Preferably, the diagnosis obtained by the method of the present invention, i.e. the diagnostic result of the method, will be used to search a database comprising recommendations of therapeutic measures for the individual possible diagnostic results.

In an embodiment, the therapy to be recommended or initiated is selected between a drug-based therapy of PCOS or for lifestyle changes to control metabolic symptoms. In embodiments, drug-based therapy of PCOS is selected from the group consisting of drugs for regulating periods, in particular oral contraceptives or progestin therapy, drugs for preventing or controlling diabetes, in particular type 2 diabetes, drugs for preventing or controlling high cholesterol, hormones or drugs to increase fertility, drugs, hormones or procedures to remove excess hair, drugs or procedure to control acne.

The methods of the present invention may be also carried out as computer-implemented inventions. In an embodiment, one or more steps, such as the comparison step and/or the calculation step are carried out by a computer comprising a processing unit (i.e. a computer). In another embodiment, all steps are carried by a computer comprising a processing unit.

Accordingly, the fourth aspect of the present invention relates to a computer-implemented method for diagnosing PCOS in subject, comprising
(a) receiving at a processing unit
   (a1) a value for the amount or concentration of LTA4H in sample from the subject and
   (a2) a value for the amount of the amount or concentration of METRNL in a sample from the subject,
(b) processing the values received in step (a) with the processing unit, wherein said processing comprises
   (b1) calculating a score of the values (a1) and (a2) received in step a),
   (b2) comparing the calculated score with a reference score and
(c) optionally providing the diagnosis via an output device, wherein said diagnosis is based on the results of step b).

In some embodiments, the processing unit is comprised by a computer.

In some embodiments, step b) according to the fourth aspect of the present invention, further comprises retrieving, at the processing unit, from a memory a reference score, i.e. a reference score which is suitable for the diagnosis of PCOS.

In an embodiment of the methods of the present invention, information on the diagnosis (according to the last step of the methods of the present invention) is provided via a display, configured for presenting the assessment. Accordingly, information may be provided whether the subject suffers from PCOS, or not, as described elsewhere herein. Further, recommendations for suitable therapeutic can be displayed. As described elsewhere herein, various therapeutic measures may be recommended. In this case, the treatment option or treatment option(s) may be shown in the display.

In an embodiment of the methods of the present invention, the methods may comprise the further step of transferring the information on the assessment of the methods of the present invention to the subject's electronic medical records.

Alternatively, the assessment made in the last step of the methods of the present invention can be printed by a printer. The print-out shall contain information on whether the patient is at risk, or not at risk and/or a recommendation of a suitable therapeutic measure.

The present invention further relates to i) the use of LTA4H and METRNL as biomarkers, or to ii) the use of at least one agent which specifically binds to METRNL and of at least one agent which specifically binds to LTA4H, for diagnosing PCOS. Preferably, said use in an in vitro use, i.e. is carried out in sample from the subject. Preferably the one agent which specifically binds to LTA4H is an antibody or an antigen-binding fragment. Preferably, the one agent which specifically binds to METRNL is an antibody or an antigen-binding fragment.

Finally, the present invention relates to a kit comprising at least one agent which specifically binds to METRNL and at least one agent which specifically binds to LTA4H.

The term “kit” as used herein refers to a collection of the aforementioned means, for example, provided in separately or within a single container. The container may comprise instructions for carrying out the method of the present invention. In one embodiment, the kit comprises reagents for the diagnosis of PCOS. The reagents of the kit may comprise antibodies or antibody fragments. Preferably, the antibodies or antibody fragments recognize epitopes or antigens of LTA4H and/or METRLN. The kit may further contain other reagents which recognize other biomarkers. Therefore, the kit may also comprise a combination of at least three reagents. According to the present invention, a biomarker can also comprise hormones, such as Anti-Müllerian Hormone, AMH. The kit can be used in any diagnostic assay.

### EXAMPLES

The invention will be merely illustrated by the following Examples. The said Examples shall, whatsoever, not be construed in a manner limiting the scope of the invention.

### Example 1: Diagnostic Performance of the Ratio Between the Two Biomarkers LTA4H and METRNL in Women with PCOS (Phenotypes A, B, C and D) and Controls Determined by ELISA Technology

Performance validation has been performed in a sample collective of 82 cases (serum samples from women with PCOS) and 44 controls (serum samples from healthy women).

The concentration of the two analytes, LTA4H and METRNL was determined by ELISA (enzyme-linked immunosorbent assay). The case group is composed of patients diagnosed with PCOS (26 phenotype A, 19 phenotype B, 20 phenotype C and 20 phenotype D) according to the Rotterdam criteria. The control group includes healthy women without PCOS.

The concentration of LTA4H in human serum was determined using the Human LTA4H ELISA kit Ver.1 from Invitrogen (catalogue number: EH308RB). The concentration of METRNL in human serum was determined using the Human METRNL ELISA kits from R&D Systems (catalog number: DY7867-05). The kits are a solid-phase sandwich Enzyme-Linked Immunosorbent Assay (ELISA) designed to detect and quantify respectively the levels of human LTA4H and METRNL in cell culture supernatants, plasma, and serum. The two analytes were measured in separate plates, only one analyte was measured at a time.

For quantitation of LTA4H, human LTA4H antibody pre-coated plates were provided with the kit. Samples were measured in 2-fold dilution. After bringing all reagents to room temperature 100 μL of each sample and standard were added. Samples and standards were measured in duplicates. During 2.5 hrs incubation at room temperature on a microplate shaker set to 650 rpm, any LTA4H present was bound to the immobilized capture antibody on the microtiter plate. During the washing step (4×300 μL), unbound substances were removed from the plate before 100 μL of the diluted anti-LTA4H biotin conjugate was added to the wells. Following 1 h incubation on a shaker and another washing step (4×300 L) to remove any unbound detection antibody, 100 μL of the prepared Streptavidin-HRP solution was added to the plate. Followed a 45 minutes incubation at room temperature and a washing step (4×300 μL). After the last wash, 100 μL of TMB Substrate was added to the plate. The plate was incubated for 30 minutes at room temperature in the dark with gentle shaking. During the incubation the substrate turned blue. The color developed in proportion to the amount of LTA4H bound in the initial step. Color development was stopped by addition of 50 μL stop solution, the solution in the well changed from blue to yellow and color intensity was measured with a plate reader at 450 nm for detection and 570 nm for background subtraction. For generation of calibration curves, lyophilized, recombinant LTA4H delivered with the kit was reconstituted and diluted in calibrator diluent. The calibration range of the assay is 2.048 ng/mL to 500 ng/mL. Calibrator 1 (500 ng/mL) corresponds to the reconstituted stock solution and calibrator 2 to calibrator 7 (2.048 ng/mL) were prepared by serial 2.5-fold dilution steps in calibrator diluent. Pure calibrator diluent served as blank (0 ng/mL). The calibration curves were fitted using a 4-parameter nonlinear regression (Newton/Raphson) with no weighting.

The capture antibody for METRNL was diluted to the working concentration in PBS without carrier protein. A 96-well microplate was incubated with 100 μL per well of the diluted Capture Antibody. The plate was sealed and incubated overnight at room temperature. The plate was then washed 3 times with 400 μL of Wash Buffer per well each time. After the last wash the Wash Buffer was completely removed, the plate was blocked by adding 300 μL of Regent Diluent to each well and incubated at room temperature for a minimum of 1 hour. The plate was washed 3 times with 400 μL of Wash Buffer per well each time and was made ready to use. Samples were measured in 4-fold dilution. After bringing all reagents to room temperature, 100 μL of each sample and standard were added. Samples and standards were measured in duplicates. During 2 hours incubation at room temperature, any METRNL present was bound to the immobilized capture antibody on the microtiter plate. During the washing steps (3×400 μL), unbound substances were removed from the plate before 100 μL of the anti-METRNL Detection Antibody diluted in Reagent Diluent was added to the wells. Following 2 hours incubation and another washing step (3×400 μL) to remove any unbound detection antibody, 100 μL of the prepared Streptavidin-HRP solution was added to the plate. Followed a 20 minutes incubation at room temperature and a washing step (3×400 μL) avoiding direct exposure to the light. After the last wash, 100 μL of Substrate Solution was added to the plate. The plate was incubated for 20 minutes at room temperature avoiding direct light exposure. During the incubation the substrate turned blue. The color developed in proportion to the amount of METRNL bound in the initial step. Color development was stopped by addition of 50 μL of Stop Solution, the color of the solution in the well changed from blue to yellow and color intensity was measured with a plate reader at 450 nm for detection and 540 or 570 nm for background subtraction. This subtraction is correcting for optical imperfections in the plate. For generation of calibration curves, lyophilized, recombinant METRNL delivered with the kit was reconstituted and diluted in the Reagent Diluent. The calibration range of the METRNL assay is 15.6 pg/mL to 1000 μg/mL. A seven point standard curve was obtained using 2-fold serial dilutions of the recombinant METRNL in the Reagent Diluent. The calibration curve was fitted using a four parameter logistic (4-PL, Newton/Raphson) curve-fit.

Once the concentrations of the two analytes were obtained by the ELISA immunoassays, the ratio LTA4H:METRNL was calculated for each sample.

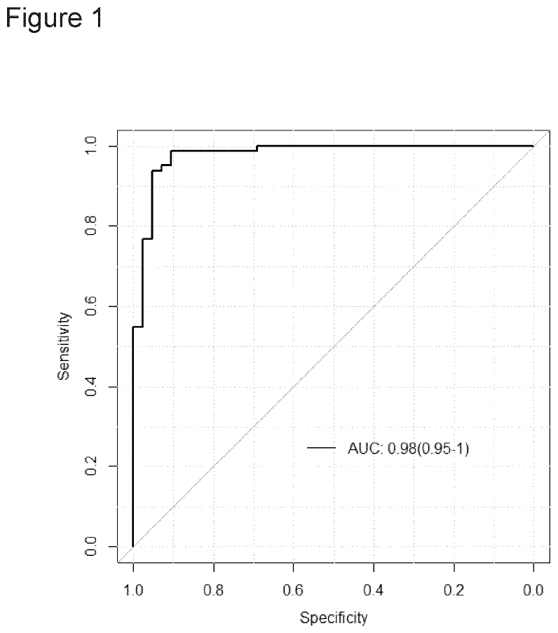

Receiver Operating Characteristic (ROC) curves were generated for the ratio LTA4H:METRNL (FIG. 1). The model performance is determined by looking at the area under the curve (AUC). The best possible AUC is 1 while the lowest possible is 0.5. The ROC curve analysis for LTA4H:METRNL ratio for PCOS cases when all phenotypes were combined (phenotypes A-D) illustrated an AUC of 0.98 (95% CI 0.95-1.00), confirming the high diagnostic accuracy of the ratio LTA4H:METRNL for PCOS (FIG. 1). The diagnostic performance of the LTA4H:METRNL ratio to distinguish between women with PCOS (cases, PCOS phenotypes A-D) and healthy control subjects using ROC analysis is shown in Table 1 describing the AUC of the ROC curve analysis and the associated 95% confidence interval.

TABLE 1

| | AUC (area | 95% CI | N | |
|---|---|---|---|---|
| | under the | (confidence | (sample | Sample |
| Marker | curve) | interval) | size) | type |
| LTA4H:METRNL | 0.98 | (0.95-1.00) | 124 | serum |

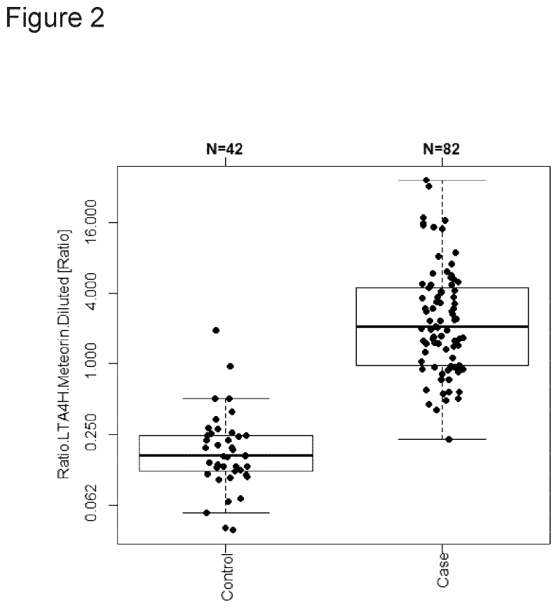

The data obtained by performing the ratio between the concentrations of LTA4H and METRNL were used to generate box and whisker plots for control and PCOS cases when all phenotypes were combined (phenotypes A-D). The LTA4H:METRNL ratio is increased in women with PCOS when compared to healthy controls (FIG. 2).

Table 2 shows the diagnostic performance of the LTA4H:METRNL ratio to distinguish between women with PCOS when separated by the different phenotypes A, B, C and D versus healthy control subjects. AUC for each phenotype is reported in the table.

TABLE 2

| | | | | | N | |
|---|---|---|---|---|---|---|
| | Pheno- | Pheno- | Pheno- | Pheno- | (sample | Sample |
| Marker | type A | type B | type C | type D | size) | type |
| LTA4H:METRNL | 0.99 | 0.99 | 0.97 | 0.97 | 124 | serum |

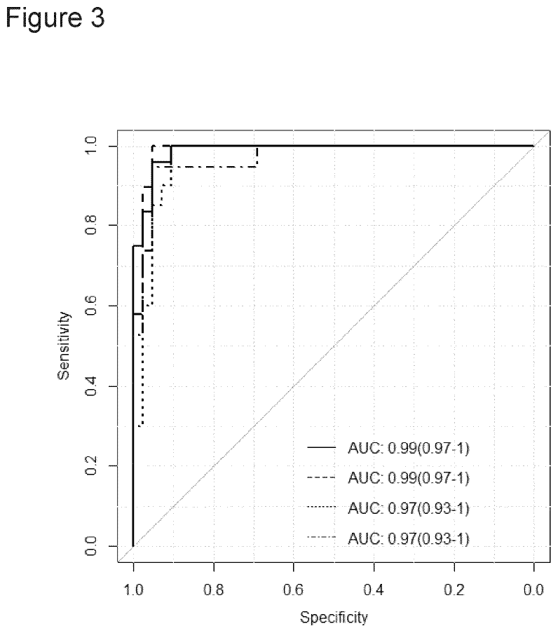

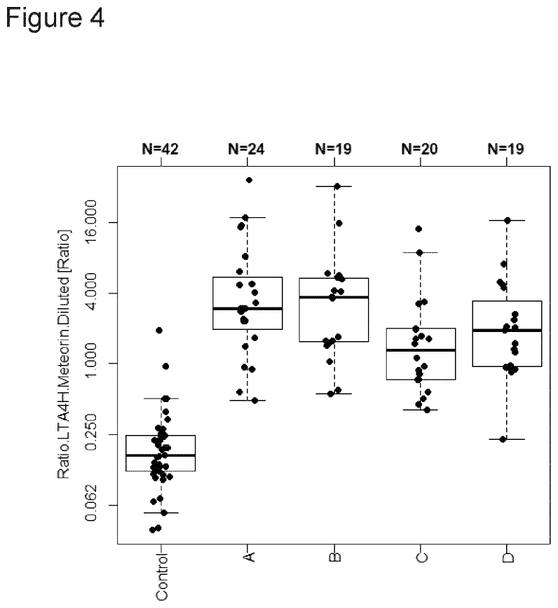

ROC curve analysis for the LTA4H:METRNL ratio for PCOS cases separated by the different phenotypes versus healthy controls showed an AUC of 0.99 (95% Cl 0.97-1.00), 0.99 (95% CI 0.97-1.00), 0.97 (95% CI 0.93-1.00) and 0.97 (95% CI 0.93-1.00), respectively for the phenotypes from A to D (FIG. 3). The results confirm the high diagnostic accuracy of the LTA4H:METRNL ratio for PCOS. The LTA4H:METRNL ratio in all the different PCOS phenotypes (Phenotype A-D) showed increased levels compared to healthy controls (FIG. 4).

Table 3 shows the diagnostic performance of the LTA4H:METRNL ratio in young women (age ≤25), to distinguish young women with PCOS from young healthy control subjects when all phenotypes were combined (phenotypes A-D).

TABLE 3

| | AUC (area | 95% CI | N | |
|---|---|---|---|---|
| | under the | (confidence | (sample | Sample |
| Marker | curve) | interval) | size) | type |
| LTA4H:METRNL | 1.00 | (1.00-1.00) | 30 | serum |

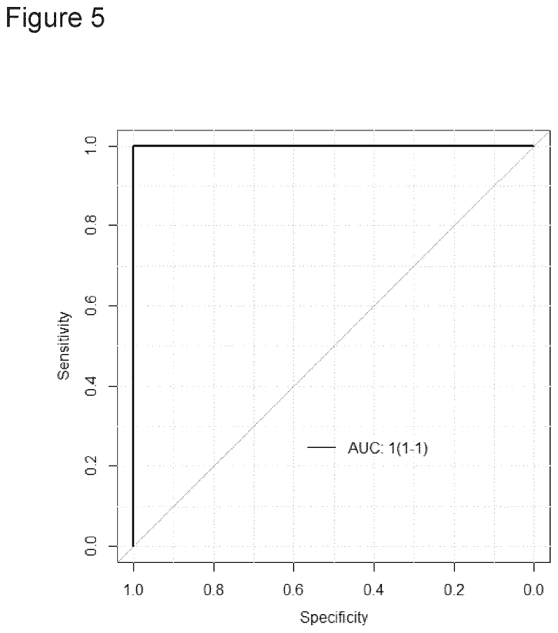

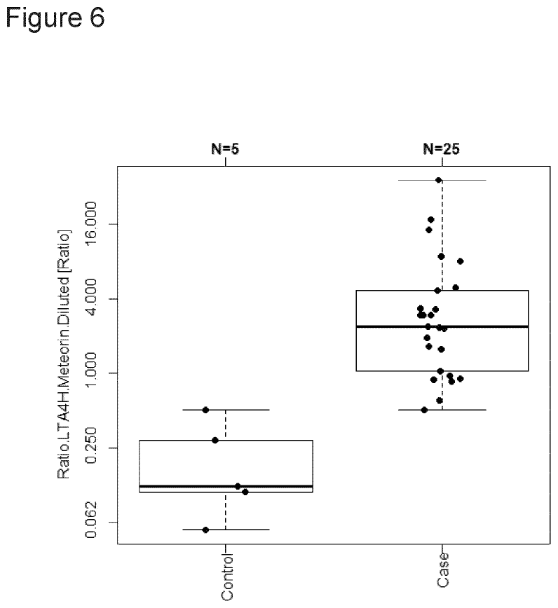

The ROC curve analysis for the LTA4H:METRNL ratio for young PCOS cases (age ≤25) when all phenotypes were combined (phenotypes A-D) illustrated an AUC of 1.00, confirming high diagnostic accuracy, for women that are 25 or younger, in discriminating PCOS cases from controls (95% CI 1-1, FIG. 5). When only young women were included in the analysis (age ≤25) PCOS cases (all phenotypes A-D combined) showed an increased LTA4H:METRNL ratio compared to young controls (age ≤25, FIG. 6).

The diagnostic performance of the LTA4H:METRNL ratio to distinguish between young women with PCOS (age ≤25) when separated by the different phenotypes A, B, C and D versus young healthy control subjects (age ≤25) has been evaluated and results are reported in table 4 (AUC for PCOS phenotypes versus controls).

TABLE 4

| | | | | | N | |
|---|---|---|---|---|---|---|
| | Pheno- | Pheno- | Pheno- | Pheno- | (sample | Sample |
| Marker | type A | type B | type C | type D | size) | type |
| LTA4H:METRNL | 1.00 | 1.00 | 1.00 | 1.00 | 30 | serum |

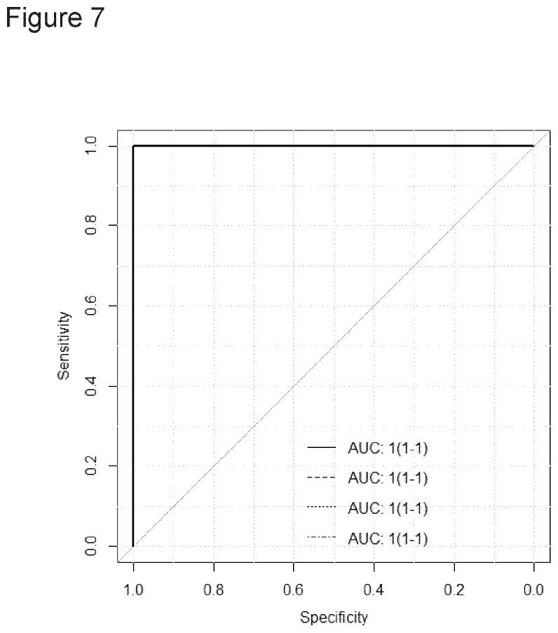

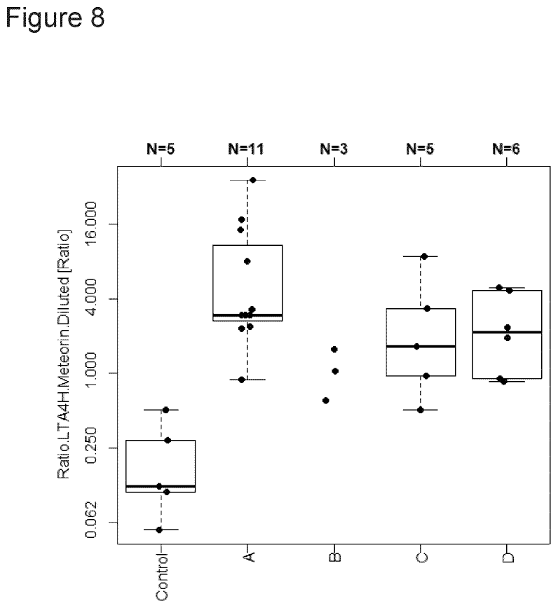

The ROC curve analysis for the LTA4H:METRNL ratio for young PCOS cases (age ≤25, phenotypes A-D) illustrated an AUC of 1.00 (95% CI 1-1) for all the phenotypes, confirming high diagnostic accuracy of the LTA4H:METRNL ratio for PCOS, in the subgroup of women of age 25 or younger (FIG. 7). The LTA4H:METRNL ratio was increased in all the different PCOS phenotypes (Phenotype A-D, age 25) when compared to the LTA4H:METRNL ratio in young healthy controls (age ≤25, FIG. 8).

### Example 2: Diagnostic Performance of the Ratio Between the Two Biomarkers LTA4H and METRNL in Women with PCOS (Phenotypes a, B, C and D) and Controls Determined by ELISA Technology, in Different Age Groups

Performance validation has been performed in an additional sample collective of 240 cases (serum samples from women with PCOS) and 48 controls (serum samples from healthy women).

The concentration of the two analytes, LTA4H and METRNL was determined by ELISA (enzyme-linked immunosorbent assay). The case group is composed of patients diagnosed with PCOS (155 phenotype A, 5 phenotype B, 8 phenotype C and 72 phenotype D) according to the Rotterdam criteria, belonging to three different age groups: 15-20 (n=70), 20-25 (n=99), 25-40 (n=71). The control group includes healthy women without PCOS.

The concentration of LTA4H in human serum was determined using the Human LTA4H ELISA kit Ver.1 from Invitrogen (catalogue number: EH308RB). The concentration of METRNL in human serum was determined using the Human METRNL ELISA kits from R&D Systems (catalog number: DY7867-05). Measurements were performed as described in example 1.

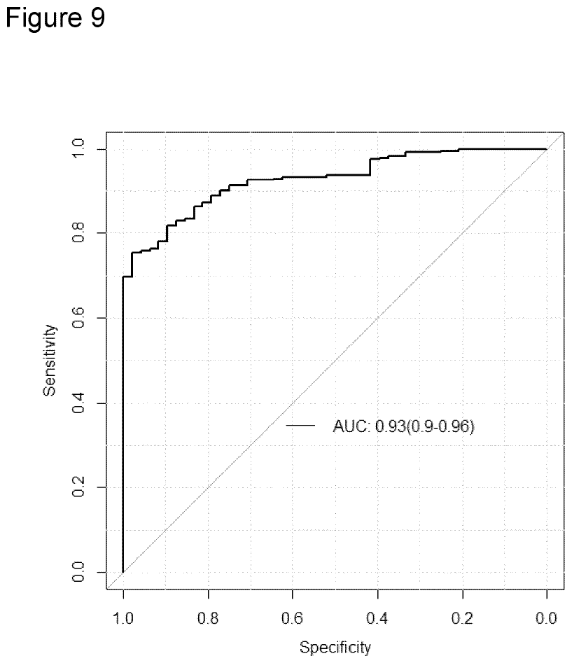

Receiver Operating Characteristic (ROC) curves were generated for the ratio LTA4H:METRNL (FIG. 9). The model performance is determined by looking at the area under the curve (AUC). The ROC curve analysis for LTA4H:METRNL ratio for PCOS cases when all phenotypes were combined (phenotypes A-D) illustrated an AUC of 0.93 (95% CI 0.90-0.96), confirming the high diagnostic accuracy of LTA4H:METRNL ratio for PCOS (FIG. 9).

The diagnostic performance of LTA4H:METRNL ratio to distinguish between women with PCOS (cases, PCOS phenotypes A-D) and healthy control subjects using ROC analysis is shown in Table 5 describing the AUC of the ROC curve analysis and the associated 95% confidence interval. The results were obtained using ELISA immunoassay.

TABLE 5

| | AUC (area | 95% CI | N | |
|---|---|---|---|---|
| | under the | (confidence | (sample | Sample |
| Marker | curve) | interval) | size) | type |
| LTA4H:METRNL | 0.93 | (0.90-0.96) | 288 | serum |

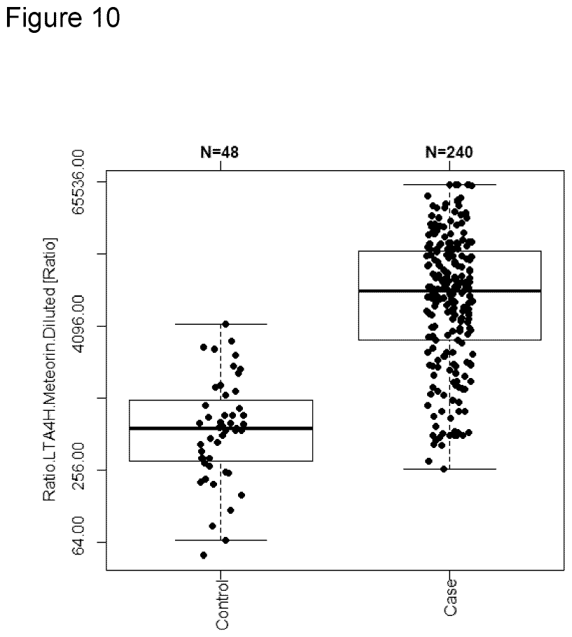

The data obtained by ELISA immunoassay were used to generate box and whisker plots for control and PCOS cases when all phenotypes were combined (phenotypes A-D). The LTA4H:METRNL ratio is increased in women with PCOS when compared to healthy controls (FIG. 10).

Table 6 shows the diagnostic performance of LTA4H:METRNL ratio to distinguish between women with PCOS when separated by the different phenotypes A, B, C and D versus healthy control subjects. AUC for each phenotype is reported in the table.

TABLE 6

| | | | | | N | |
|---|---|---|---|---|---|---|
| | Pheno- | Pheno- | Pheno- | Pheno- | (sample | Sample |
| Marker | type A | type B | type C | type D | size) | type |
| LTA4H:METRNL | 0.93 | 1.00 | 0.93 | 0.92 | 288 | serum |

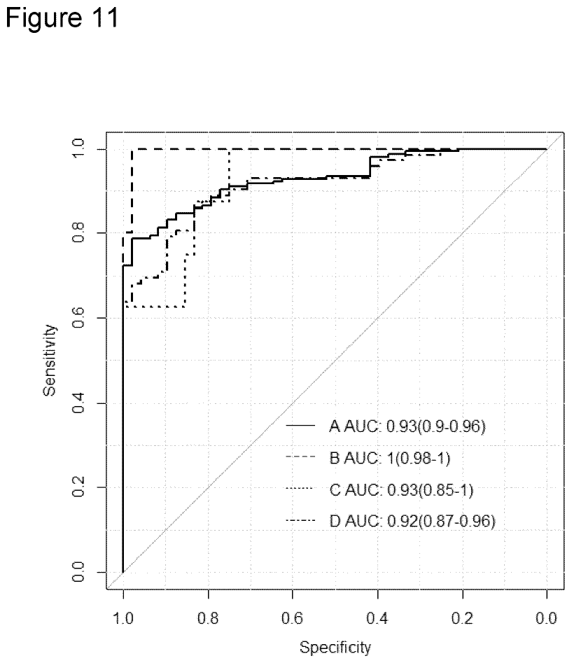

ROC curve analysis for LTA4H:METRNL ratio for PCOS cases separated by the different phenotypes versus healthy controls showed an AUC of 0.93 (95% CI 0.90-0.96), 1.00 (95% CI 0.98-1.00), 0.93 (95% CI 0.85-1.00) and 0.92 (95% CI 0.87-0.96), respectively for the phenotypes from A to D (FIG. 11). The results confirm the high diagnostic accuracy of LTA4H:METRNL ratio for PCOS.

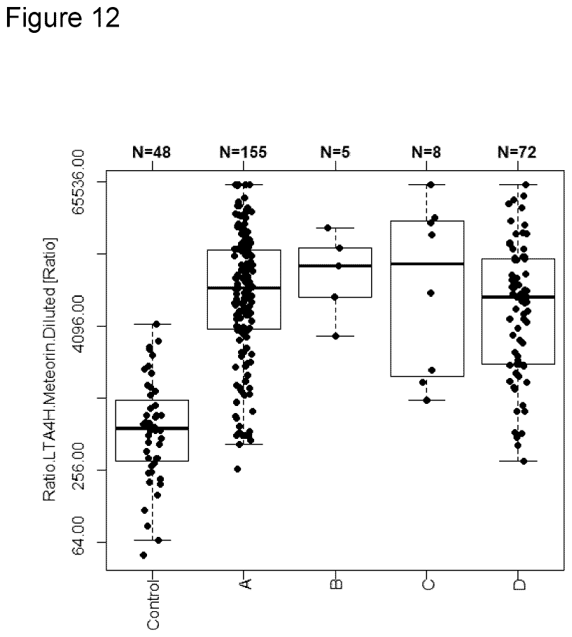

The LTA4H:METRNL ratio in all the different PCOS phenotypes (Phenotype A-D) was increased compared to healthy controls (FIG. 12).

Table 7 shows the diagnostic performance of LTA4H:METRNL ratio in different age groups (15≤age <20, 20≤age <25, 25≤age <40), to distinguish women with PCOS from healthy control subjects when all phenotypes were combined (phenotypes A-D).

TABLE 7

| | 15 ≤ | 20 ≤ | 25 ≤ | N | |
|---|---|---|---|---|---|
| | age < | age < | age < | (sample | Sample |
| Marker | 20 | 25 | 40 | size) | type |
| LTA4H:METRNL | 0.91 | 0.91 | 0.97 | 288 | serum |

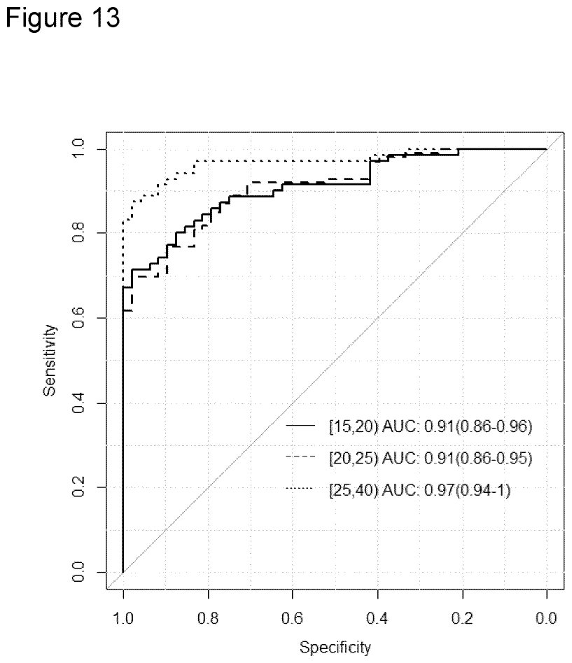

ROC curve analysis for LTA4H:METRNL ratio for PCOS cases separated by the different age groups versus healthy controls showed an AUC of 0.91 (95% CI 0.86-0.96), 0.91 (95% CI 0.86-0.95) and 0.97 (95% CI 0.94-1.00), respectively for the age groups 15-20, 20-25, 25-40 (FIG. 13). The results confirm the high diagnostic accuracy of LTA4H:METRNL ratio for PCOS in all the different age groups.

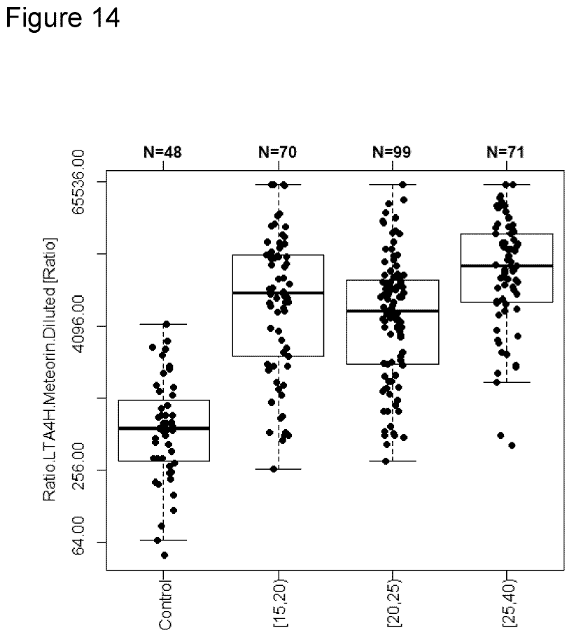

Increased LTA4H:METRNL ratio in women with PCOS compared to controls was confirmed in all the different age groups (FIG. 14).

Considering the lack of reliable biomarkers for diagnosing PCOS especially in young women (age <25), a separate analysis was performed for the age group ≥15 and <25.

Table 8 shows the diagnostic performance of LTA4H:METRNL ratio in young women (15≤age <25), to distinguish young women with PCOS from young healthy control subjects when all phenotypes were combined (phenotypes A-D).

TABLE 8

| | AUC (area | 95% CI | N | |
|---|---|---|---|---|
| | under the | (confidence | (sample | Sample |
| Marker | curve) | interval) | size) | type |
| LTA4H:METRNL | 0.92 | (0.87-0.97) | 192 | serum |

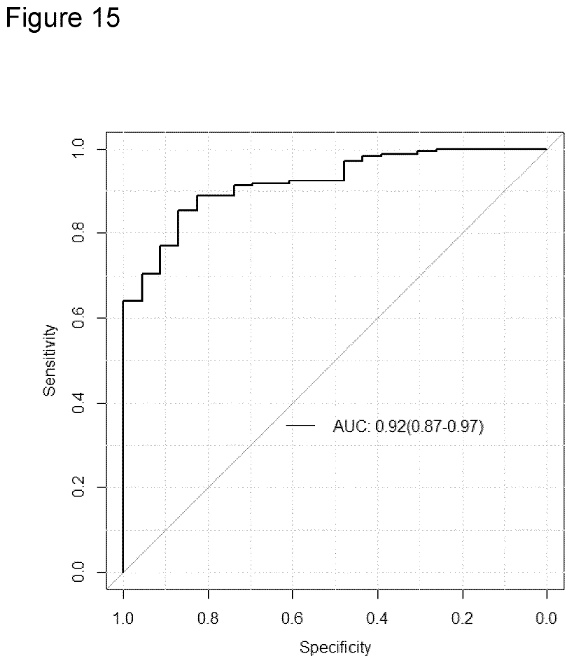

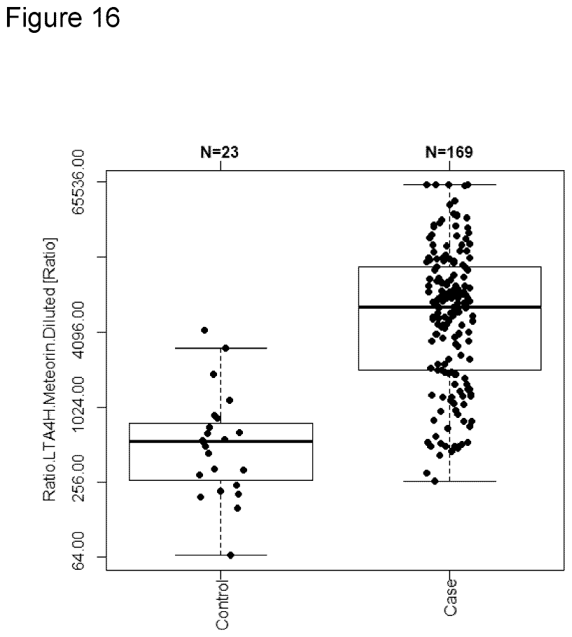

The ROC curve analysis for the LTA4H:METRNL ratio for young PCOS cases (15≤age <25) when all phenotypes were combined (phenotypes A-D) illustrated an AUC of 0.92, confirming high diagnostic accuracy for 15-25 year old women, in discriminating PCOS cases from controls (95% CI 0.87-0.97, FIG. 15). When only young women were included in the analysis (age 15-25) PCOS cases (all phenotypes A-D combined) showed increased LTA4H:METRNL ratio compared to young controls (15≤age <25, FIG. 16).

The diagnostic performance of the LTA4H:METRNL ratio to distinguish between young women with PCOS (15≤age <25) when separated by the different phenotypes A, B, C and D versus young healthy control subjects (15≤age <25) has been evaluated and results are reported in table 9 (AUC for PCOS phenotypes versus controls).

TABLE 9

| | | | | | N | |
|---|---|---|---|---|---|---|
| | Pheno- | Pheno- | Pheno- | Pheno- | (sample | Sample |
| Marker | type A | type B | type C | type D | size) | type |
| LTA4H:METRNL | 0.92 | 1.00 | 0.94 | 0.91 | 192 | serum |

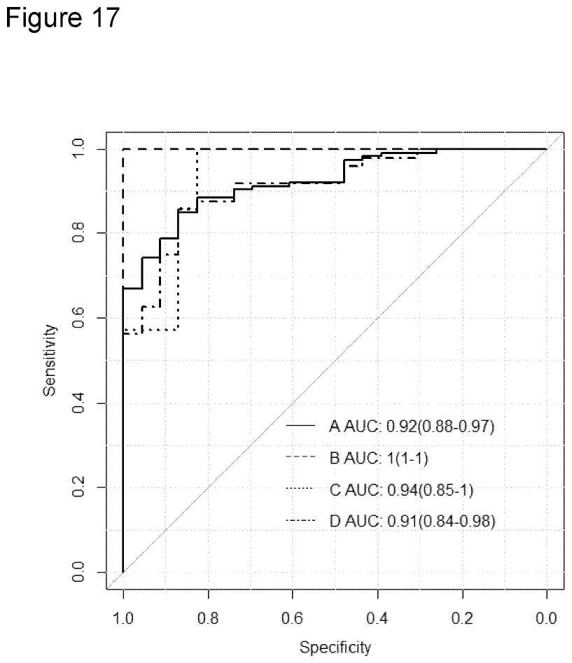

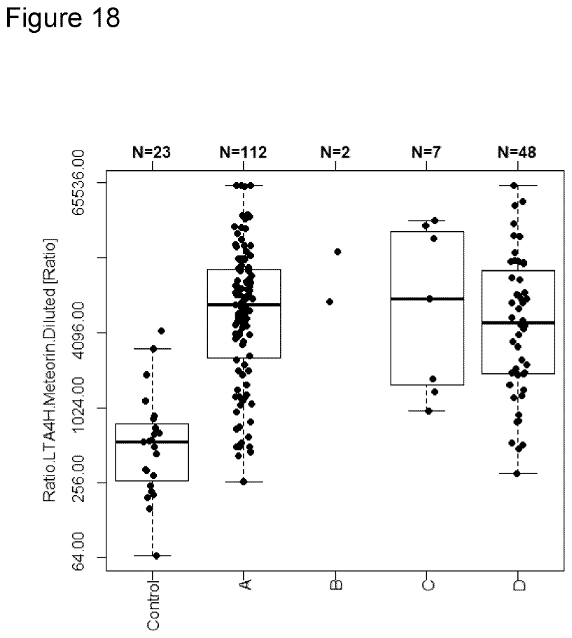

The ROC curve analysis for LTA4H:METRNL ratio for young PCOS cases (15≤age <25, phenotypes A-D) illustrated an AUC of 0.92 (95% CI 0.88-0.97), 1.00 (95% CI 1.00-1.00), 0.94 (95% CI 0.85-1.00), 0.91 (95% CI 0.84-0.98) for each phenotype respectively, confirming high diagnostic accuracy of the LTA4H:METRNL ratio for PCOS, in the subgroup of women of age 15-25 (FIG. 17). The LTA4H:METRNL ratio was increased in all the different PCOS phenotypes (Phenotype A-D, 15≤age <25), when compared to the LTA4H:METRNL ratio in young healthy controls (15≤age <25, FIG. 18).

1. A method for diagnosing PCOS in a subject, said method comprising the steps of
a) determining the amount or concentration of LTA4H in a sample from the subject;
b) determining the amount or concentration of METRNL in a sample from the subject;
c) calculating a score of the amounts or concentrations determined in steps a) and b);
d) comparing the calculated score with a reference score; and
e) diagnosing PCOS in a subject.

2. A method of selecting a patient for therapy of PCOS, said method comprising:
a) determining the amount or concentration of LTA4H in a sample from the subject;
b) determining the amount or concentration of METRNL in a sample from the subject;
c) calculating a score of the amounts or concentrations determined in steps a) and b);
d) comparing the calculated score with a reference score; and
e) selecting the patient for PCOS therapy.

3. A method for monitoring PCOS progression in a subject having PCOS or for monitoring response to treatment in a subject having PCOS, said method comprising
a) determining the amount or concentration of LTA4H in a sample from the subject;
b) determining the amount or concentration of METRNL in a sample from the subject;
c) calculating a score of the amounts or concentrations determined in steps a) and b);
d) comparing the calculated score with a reference score; and
e) monitoring progression in the subject suffering from PCOS or being treated for PCOS.

4. The method of claim 1, wherein the sample is blood, serum, plasma, capillary blood, interstitial fluid, peritoneal fluid or menstrual fluid, and/or wherein the subject is a human subject.

5. The method of claim 1, wherein the subject is suspected to suffer from PCOS, and wherein the subject who is suspected to suffer from PCOS has a history of PCOS symptoms.

6. The method of claim 1, wherein the determination of the amount or concentration of LTA4H comprises contacting the sample with an antibody, or antigen-binding fragment thereof, which specifically detects LTA4H.

7. The method of claim 1, wherein the determination of the amount or concentration of METRNL comprises contacting the sample with an antibody, or antigen-binding fragment thereof, which specifically detects METRNL.

8. The method of claim 1, wherein the score is a ratio,
wherein the ratio is the ratio of the amount or concentration of LTA4H to the amount or concentration of METRNL, and wherein a ratio which is higher than the reference ratio is indicative for a subject who suffers from PCOS, or
wherein the ratio is the ratio of the amount or concentration of METRNL to the amount or concentration of LTA4H, and wherein a ratio which is lower than the reference ratio is indicative for a subject who suffers from PCOS.

9. The method of claim 1, wherein PCOS is metabolic or phenotypical PCOS.

10. The method of claim 1, wherein PCOS is selected from the group consisting of phenotype A, phenotype B, phenotype C and phenotype D.

11. (canceled)

12. (canceled)

13. A kit comprising at least one agent which specifically binds to METRNL and at least one agent which specifically binds to LTA4H.

14. The method of claim 1, wherein the reference score is derived from a sample from a subject, or from samples from a group of subjects, known to suffer from PCOS or known not to suffer from PCOS.

15. The method of claim 2, wherein the therapy is a drug-based therapy of PCOS or lifestyle changes to control metabolic symptoms.

16. The method of claim 15, wherein the drug-based therapy of PCOS is selected from the group consisting of drugs for regulating periods, drugs for preventing or controlling diabetes, drugs for preventing or controlling high cholesterol, hormones or drugs to increase fertility, hormones to remove excess hair, and drugs to control acne.

17. The method of claim 16, wherein the drugs for regulating periods are oral contraceptives or progestin therapy.

18. The kit of claim 13, wherein the at least one agent which specifically binds to METRNL is an antibody, or an antigen-binding fragment thereof, and wherein the at least one agent which specifically binds to LTA4H is an antibody, or an antigen-binding fragment thereof.

19. The method of claim 6, wherein the determination of the amount or concentration of LTA4H comprises performing an immunoassay, and wherein the immunoassay is selected from the group consisting of an enzyme linked immunosorbent assay (ELISA), an enzyme immunoassay (EIA), a radioimmunoassay (RIA), and an immuno assay based on detection of luminescence, fluorescence, chemiluminescence or electrochemiluminescence.

20. The method of claim 1, wherein step a) comprises:
i) incubating the sample of the subject with one or more antibodies specifically binding to LTA4H, thereby generating a complex between the antibody and LTA4H,
ii) incubating the sample of the subject with one or more antibodies specifically binding to METRNL, thereby generating a complex between the antibody and METRNL, and
iii) quantifying the complexes formed in steps i) and ii), thereby quantifying the amount or concentration of LTA4H and METRNL in the sample of the subject.

21. The method of claim 20, wherein in step i), the sample is incubated with two antibodies specifically binding to LTA4H.

22. The method of claim 21, wherein a sandwich will be formed comprising a first antibody to LTA4H, LTA4H (analyte) and the second antibody to LTA4H, wherein the second antibody is detectably labeled.
